(19) United States
(12) Patent Application Publication
(10) Pub. No.: US 2009/0081631 A1
(43) Pub. Date: Mar. 26, 2009

(54) Tissue Engineered Organ

(76) Inventor: Clay David Fette, Palm Beach Gardens, FL (US)

Correspondence Address: Clay Fette, 2322 Dawn Drive, Crete, NE, 68333, US

(21) Appl. No.: 11/858,156

(22) Filed: Sep. 20, 2007

(51) Int. Cl. A61P 43/00; A01N 1/00

(52) U.S. Cl. 435/1.1

(57) ABSTRACT

Tissue engineered organs and methods for producing the same are provided. The tissue engineered organs are useful to replace diseased or damaged host organs.

### FIELD OF THE INVENTION

The present invention relates in general to the field of medical devices. More particularly, the present invention relates to tissue engineering and methods.

### BACKGROUND OF THE INVENTION

Many people suffer from disease or injury that requires an organ transplant. These individuals suffering from damaged or diseased organs must currently wait for a compatible donor organ to become available. After this often lengthy process they also must then worry about rejection, and take anti-rejection medication for the rest of their lives, as well as undergo a series of checkups and biopsies to assess the status of the donor organ. New solutions that can overcome these issues are desirable.

Current processed allograft tissue products include cryopreserved blood vessels and heart valves, as well a variety of processed bone materials. Current processed xenograft tissue products are more numerous, such as processed bovine or porcine collagen products. Xenograft products produced which maintain their native architecture are lesser in number, however. Bovine bone products are one example. Some products manufactured from porcine small intestine submucosa are another example. There are also currently living cell products available, such as DermaGraft® and TransCyte®. However, none of these products employ the use of more than one cell type, nor do they combine a cell type with a tissue material of complex native architecture.

Large implantable tissue materials have difficulty with cell migration due to the distance needed to travel, as well as difficulty with vascularization after implantation. Polymer, hydrogel, collagen and other pre-formed scaffolds are difficult to manufacture mimicking the native architecture of the host organ.

Given the limitations with currently available products and tissue engineering scaffolds to treat diseased or damaged organs, it would be desirable to develop a tissue engineered organ-like structure that could overcome these limitations.

### BRIEF SUMMARY OF THE INVENTION

The proposed invention provides the methods for achieving creation of an organ-like structure. This created structure would be used as a replacement for a diseased or damaged host organ. This created structure would have minimal to no compatibility or rejection issues as seen in current donor source organ transplants.

The proposed invention overcomes deficiencies in the prior art by using processed allograft or xenograft tissue as the basis for cell culture. Using this tissue provides the native architecture and structure that is seen in the host organ targeted for replacement.

The proposed invention also overcomes defects in the prior art through the use of the addition of cells to the processed tissue. This addition of cells increases the durability, functionality, and viability of the structure. It will also help provide the necessary vascularization.

Organ-like structures produced by the methods described herein are also encompassed by the present invention.

These and other objects and embodiments are described in or are obvious from the following Detailed Description and are within the scope of the invention.

### DETAILED DESCRIPTION OF THE INVENTION

The present methods comprise the use of a tissue material in combination with a plurality of cell types to develop an organ-like structure. The tissue material can be comprised of allograft or xenograft material. This tissue material is processed in such a way as to render it free of viable cells. This tissue material is then combined with a plurality of cell types to create an organ-like structure. This organ-like structure can then be implanted to replace a diseased or damaged host organ.

As used herein, the terms “comprises”, “comprising” and the like can have the meaning ascribed to them in U.S. Patent Law and can mean “includes, “including” and the like.

In the context of this application, “organ-like structure” comprises a structure that has full or partial function of the native organ it resembles. Examples would include a structure resembling and functioning similar to a kidney, a liver, a pancreas, a heart, an intestine, a lung.

Preferably, the allograft organ or tissue is harvested from a human source by currently approved methods and guidelines. The allograft organ or tissue is then processed according to standard industry practice, to result in no viable cells remaining, to remove viruses, and to remove other undesirable components. The resultant tissue material is free of living cells and maintains the natural architecture and structure of the original organ or tissue. Examples of treatment include detergents, salt solutions, alcohols, antibiotics, and antimicrobials.

The allograft organs or tissue contained in this embodiment include kidney, liver, pancreas, heart, intestine and lung.

The processed allograft organ or tissue is then further developed in cell culture. Common methods and materials can be employed to accomplish the cell culture applications, such as cell seeding, media preparation, oxygenation, and nutrient addition. Specific cell types are to be used in culture. Examples of the specific cell types are renal cells, hepatocytes, islet cells, myocytes, enterocytes and pneumocytes.

The processed allograft material is combined with the cells in culture in a manner which will direct the cells and the culture media to the desired locations or areas within the allograft material. This will allow for specific cells types to be cultured in specific areas or sections of the processed allograft material along with a media of specific composition, while a different collection of cell types and a different specific media could be cultured in different specific areas and sections of the processed allograft material.

The resultant organ-like structure will be composed of allograft organ or tissue and a plurality of cell types. The organ-like structure in this embodiment includes kidney, liver, pancreas, heart, intestine, and lung.

An example of this embodiment would be the combination of renal cells and media being exposed or cultured only onto and into the main body of the processed allograft kidney tissue while vascular cell types such as endothelium and media would be exposed to only the vascular network of the processed allograft kidney. Urothelial cells could also simultaneously be cultured in the ureter or urology-related anatomy with a third media and flow system.

Another example of this embodiment would be the combination of hepatocytes and media being exposed or cultured only onto and into the main body of the processed allograft liver tissue while vascular cell types such as endothelium and media would be exposed to only the vascular network of the processed allograft liver.

Another example of this embodiment would be the combination of islet cells and media being exposed or cultured only onto and into the main body of the processed allograft pancreas tissue while vascular cell types such as endothelium and media would be exposed to only the vascular network of the processed allograft pancreas.

Another example of this embodiment would be the combination of myocytes and media being exposed or cultured only onto and into the main body of the processed allograft heart tissue while vascular cell types such as endothelium and media would be exposed to only the vascular network of the processed allograft heart. Nerve cells could also be simultaneously cultured in the original nerve areas of the heart with a third media and flow system.

Another example of this embodiment would be the combination of enterocytes and media being exposed or cultured only onto and into the main body of the processed allograft intestine tissue while vascular cell types such as endothelium and media would be exposed to only the vascular network of the processed allograft intestine. Muscle cells could also be simultaneously cultured in the original muscle areas of the intestine with a third media and flow system.

Another example of this embodiment would be the combination of pneumocytes and media being exposed or cultured only onto and into the main body of the processed allograft lung tissue while vascular cell types such as endothelium and media would be exposed to only the vascular network of the processed allograft lung.

Another example of this embodiment would be the combination of stem cells with any of the aforementioned allograft tissue materials. The stem cells could be used in combination with any of the aforementioned cell types, either concurrently in the same specific tissue area, or in different areas of the tissue.

Preferably, the xenograft organ or tissue is harvested from a human source by currently approved methods and guidelines. The xenograft organ or tissue is then processed according to standard industry practice, to result in no viable cells remaining, to remove viruses, and to remove other undesirable components. The resultant tissue material is free of living cells and maintains the natural architecture and structure of the original organ or tissue. Examples of treatment include detergents, salt solutions, alcohols, antibiotics, and antimicrobials.

The xenograft organs or tissue contained in this embodiment include kidney, liver, pancreas, heart, intestine and lung.

The processed xenograft organ or tissue is then further developed in cell culture. Common methods and materials can be employed to accomplish the cell culture applications, such as cell seeding, media preparation, oxygenation, and nutrient addition. Specific cell types are to be used in culture. Examples of the specific cell types are renal cells, hepatocytes, islet cells, myocytes, enterocytes and pneumocytes.

The processed xenograft material is combined with the cells in culture in a manner which will direct the cells and the culture media to the desired locations or areas within the xenograft material. This will allow for specific cells types to be cultured in specific areas or sections of the processed xenograft material along with a media of specific composition, while a different collection of cell types and a different specific media could be cultured in different specific areas and sections of the processed xenograft material.

The resultant organ-like structure will be composed of xenograft organ or tissue and a plurality of cell types. The organ-like structure in this embodiment includes kidney, liver, pancreas, heart, intestine, and lung.

An example of this embodiment would be the combination of renal cells and media being exposed or cultured only onto and into the main body of the processed xenograft kidney tissue while vascular cell types such as endothelium and media would be exposed to only the vascular network of the processed xenograft kidney. Urothelial cells could also simultaneously be cultured in the ureter or urology-related anatomy with a third media and flow system.

Another example of this embodiment would be the combination of hepatocytes and media being exposed or cultured only onto and into the main body of the processed xenograft liver tissue while vascular cell types such as endothelium and media would be exposed to only the vascular network of the processed xenograft liver.

Another example of this embodiment would be the combination of islet cells and media being exposed or cultured only onto and into the main body of the processed xenograft pancreas tissue while vascular cell types such as endothelium and media would be exposed to only the vascular network of the processed xenograft pancreas.

Another example of this embodiment would be the combination of myocytes and media being exposed or cultured only onto and into the main body of the processed xenograft heart tissue while vascular cell types such as endothelium and media would be exposed to only the vascular network of the processed xenograft heart. Nerve cells could also be simultaneously cultured in the original nerve areas of the heart with a third media and flow system.

Another example of this embodiment would be the combination of enterocytes and media being exposed or cultured only onto and into the main body of the processed xenograft intestine tissue while vascular cell types such as endothelium and media would be exposed to only the vascular network of the processed xenograft intestine. Muscle cells could also be simultaneously cultured in the original muscle areas of the intestine with a third media and flow system.

Another example of this embodiment would be the combination of pneumocytes and media being exposed or cultured only onto and into the main body of the processed xenograft lung tissue while vascular cell types such as endothelium and media would be exposed to only the vascular network of the processed xenograft lung.

Another example of this embodiment would be the combination of stem cells with any of the aforementioned xenograft tissue materials. The stem cells could be used in combination with any of the aforementioned cell types, either concurrently in the same specific tissue area, or in different areas of the tissue.

While the invention has been described in detail with reference to preferred embodiments thereof, it will be apparent to one skilled in the art that various changes can be made, and equivalents employed, without departing from the scope of the invention.

I claim:

1. The method of using tissue material in combination with a plurality of cell types to develop an organ-like structure.

2. The method of claim 1 wherein the tissue material is allograft kidney.

3. The method of claim 1 wherein the tissue material is allograft liver.

4. The method of claim 1 wherein the tissue material is allograft pancreas.

5. The method of claim 1 wherein the tissue material is allograft heart.

6. The method of claim 1 wherein the tissue material is allograft intestine.

7. The method of claim 1 wherein the tissue material is allograft lung.

8. The method of claim 1 wherein the tissue material is xenograft kidney.

9. The method of claim 1 wherein the tissue material is xenograft liver.

10. The method of claim 1 wherein the tissue material is xenograft pancreas.

11. The method of claim 1 wherein the tissue material is xenograft heart.

12. The method of claim 1 wherein the tissue material is xenograft intestine.

13. The method of claim 1 wherein the tissue material is xenograft lung.

14. The method of claim 1 wherein at least one of the cell types is renal cells.

15. The method of claim 1 wherein at least one of the cell types is hepatocytes.

16. The method of claim 1 wherein at least one of the cell types is islet cells.

17. The method of claim 1 wherein at least one of the cell types is myocytes.

18. The method of claim 1 wherein at least one of the cell types is enterocytes.

19. The method of claim 1 wherein at least one of the cell types is pneumocytes

20. The method of claim 1 wherein at least one of the cell types is stem cells.

21. An organ-like structure comprising allograft tissue and a plurality of cell types.

22. An organ-like structure comprising xenograft tissue and a plurality of cell types.
